(19) United States
(12) Patent Application Publication
(10) Pub. No.: US 2017/0127927 A1
(43) Pub. Date: May 11, 2017

(54) Light-Conducting Device for an Endoscope to Direct Illuminating Light

(71) Applicant: Karl Storz GmbH & Co. KG, Tuttlingen (DE)

(72) Inventors: Fang Lei, Durchhausen (DE); Ulrich Weiger, Kolbingen (DE); Andre Buerk, Villingen-Schwenningen (DE)

(21) Appl. No.: 15/411,588

(22) Filed: Jan. 20, 2017

Related U.S. Application Data
(63) Continuation of application No. 13/452,158, filed on Apr. 20, 2012.

(30) Foreign Application Priority Data
Apr. 21, 2011 (DE) 102011007880.0

(51) Int. Cl. A61B 1/07; A61B 1/00; G02B 6/04; G02B 23/24; F21V 8/00

(52) U.S. Cl. CPC A61B 1/07; G02B 23/2469; G02B 6/0008; G02B 6/04; A61B 1/0011

(57) ABSTRACT

A light-conducting device for an endoscope to conduct illuminating light to the distal end of the endoscope including a curved, rigid portion with a predetermined spatial configuration, such that the curved, rigid portion is foreseen for positioning on a distal end of an endoscope, such that the curved, rigid portion has its rigid property at least either before insertion of the light-conducting device into an endoscope or before configuration of a direct or indirect mechanical connection of the light-conducting device with an inner shaft for an endoscope or before configuration of a direct or indirect mechanical connection of the light-conducting device with an outer shaft for an endoscope.

### CROSS-REFERENCE TO RELATED APPLICATIONS

This application is a continuation of Ser. No. 13/452,158 filed on Apr. 20, 2012, which claimed priority of German patent application No. 10 2011 007 880.0 filed on Apr. 21, 2011.

### FIELD OF THE INVENTION

The present invention relates to a light-conducting device for an endoscope to direct illuminating light to the distal end of the endoscope, to an endoscope with a light-conducting device and also to a method for producing a light-conducting device and for producing an endoscope.

### BACKGROUND OF THE INVENTION

Illumination of the observed object is necessary as a rule in medical and technical endoscopy. To generate illuminating light with a high light beam and desired spectral properties, in particular with good color reproduction, use is often made of light source devices that are separate or integrated into the proximal end of the endoscope. The illuminating light is transmitted from the proximal to the distal end of the endoscope by means of one or more bundles of optic fibers.

In constructing and manufacturing endoscopes, considerable expense is required for precise positioning of the optic fibers, in particular of their distal ends. In manufacturing endoscopes there is a marked tendency toward constantly thinner shafts. Thus, there is always less construction space available for the optic fibers. Consequently, there is a rise in construction expense required to maintain the necessary minimum curvature radii for optic fibers. Above all, there is an increase in the expenditure for manufacturing and the generated scrap, because constantly declining numbers of optic fibers are being curved more and more strongly.

### SUMMARY OF THE INVENTION

It is an object of the present invention to provide an improved light-conducting device to direct illuminating light to a distal end of an endoscope, an improved endoscope and improved methods for producing a light-conducting device and an endoscope.

This object is achieved by means of the contents of the independent claims.

Refinements are indicated in the dependent claims.

Embodiments of the present invention are based on the idea of not using loose bundles of optic fibers in manufacturing an endoscope, but rather a light-conducting device with a curved rigid portion on the distal end. The light-conducting device pre-formed in this manner can thus be produced outside of the endoscope and thus at a distance in terms of space and time and logistically independently, with its function tested. As a rigid unit, the distal end of the light-conducting device in particular has an increased mechanical robustness. The risk of damaging the light-conducting device in inserting it into the shaft of the future endoscope is thereby markedly reduced.

A light-conducting device for an endoscope to direct illuminating light to the distal end of the endoscope, even before insertion of an inner shaft into an outer shaft for the endoscope, has a curved, rigid portion with a predetermined spatial configuration.

A light-conducting device for an endoscope to direct illuminating light to the distal end of the endoscope comprises a curved, rigid portion with a predetermined spatial configuration, such that the curved, rigid portion is foreseen for positioning on a distal end of an endoscope, such that the curved, rigid portion already has its rigid property at least either before an insertion of the light-conducting device into an endoscope or before configuration of a direct or indirect mechanical connection of the light-conducting device with an outer shaft for an endoscope.

A curvature of the light-conducting device, in the sense of the present invention, is a curvature of the propagation path foreseen for the illuminating light inside the light-conducting device. The propagation path foreseen for the illuminating light is represented in particular by the smooth line that is formed by the surface center points of the cross-section surfaces of the light-conducting device, such that the cross-section surfaces are, in particular, perpendicular to the line. For example, with a Gauss-shaped distribution of the light flow from illuminating light switched into the light-conducting device, the sites of maximum light flow density or maximum intensity are situated as a rule on this line or close to this line.

The light-conducting device comprises the curved, rigid portion even before the temporary or definitive insertion of the inner shaft and in particular also before the temporary insertion of a dummy inner shaft in the outer shaft. The curved, rigid portion of the light-conducting device is therefore generated, in particular, outside the outer shaft and, in particular, at some distance, in time and space, from the manufacture of other components of the endoscope.

The separation from the manufacture of other components of the endoscope, in terms of time, space and thus also logistics, can reduce production costs. In addition, the light-conducting device can be tested before insertion, so that because of the rigidity of the curved portion, there is less damage on inserting—for example, compared with the insertion of a flexible bundle of optic fibers.

The rigid portion of the light-conducting device can include the entire light-conducting device and thus can extend from the light inlet surface all the way to the light outlet surface of the light-conducting device. Alternatively, the rigid portion can include just one part of the light-conducting device, so that the rigid portion in particular borders on the light outlet surface or includes the area of the light-conducting device in which a light outlet surface is generated after inserting the light-conducting device into an outer shaft for an endoscope. The rigid portion, in addition to one or more curved areas, can include one or more straight portions.

The predetermined spatial configuration of the curved, rigid portion, in addition to one or more curvatures with one or more fixed or continuously or discontinuously varying curvature radii, can also include transitions between various cross-section surfaces with constant or varying surface area.

The light-conducting device is configured in particular for an endoscope with a fixed or adjustable viewing angle that is not parallel to the longitudinal axis of the endoscope. In particular at an angle greater than 30 degrees or 45 degrees between the viewing angle and the longitudinal axis of the endoscope, the required corresponding curvature of the light-conducting device gives rise to the problems described above, which can be partly or completely solved with the inventive light-conducting device.

The inner shaft includes, in particular, the observation beam path with an objective lens or other imaging device, a rod lens telescope or an arranged bundle of optic fibers or an image sensor. The light-conducting device, in particular, is provided for positioning in an intermediate space between the inner shaft and the outer shaft.

The light-conducting device with the curved, rigid portion can therefore be configured as a completely separate component or as a component that is partly or completely joined with the inner shaft.

A light-conducting device as described here includes in particular a number of light-conducting devices that are joined together in the rigid portion of the light-conducting device and are curved.

Light transmission is possible alternatively, for example, in thin rods of transparent material. The light-conducting property, however, can be partly or completely lost if the ratio between the curvature radius and the diameter of the rod is not large enough. At small curvature radii, therefore, the thinnest possible optic fibers in comparison with light-conducting rods or thick optic fibers can have the advantage of a markedly smaller loss in illuminating light. Alternatively, the light-conducting device comprises a layered structure, a superlattice or other inner structure, which supports light transmission along a foreseen curved propagation path.

The optic fibers are in particular welded together or pressed together at a temperature close to the melting point or glass transition temperature or refractory deformation temperature. This allows an especially robust firmly bonded connection. In soldering or heat-pressing the optic fibers, in addition, intervals between the optic fibers can be removed. Thereby, in particular, originally circular cross-sections of the individual optic fibers become hexagonal or otherwise polygonal in shape. Consequently, the curved, rigid portion can form a fluid-tight portion of a surface of an endoscope that also allows no penetration of moisture in autoclaving the endoscope. Alternatively, the optic fibers can be soldered, cemented or cast with a metallic or non-metallic solder so that the curved, rigid portion can likewise be fluid-tight.

With a light-conducting device as described here, with a number of mutually joined optic fibers, the individual optic fibers can each comprise a cross-section with a surface area that varies in the light propagation direction.

By varying the cross-sections of the individual optic fibers, it is possible to adjust the divergence or solid angle into which illuminating light emerges at the light outlet surfaces of the optic fibers. Thus, in particular, the solid angle into which the illuminating light emerges can be adjusted to the site of the visual field.

With a light-conducting device as described here, the surface normals of a light inlet surface and of a light outlet surface of the light-conducting device are, in particular, not parallel.

In the case of a curved light inlet surface or a curved light outlet surface, the surface normals are understood to mean the center surface normal or the surface normal in the center of the respective surface. The surface normal of the light inlet surface can be parallel to the foreseen light propagation direction in the light-conducting device directly downstream in the light path from the light inlet surface. The surface normal of the light outlet surface is, in particular, parallel to the light propagation direction in the light-conducting device directly upstream in the light path from the light outlet surface. The surface normal of the light inlet surface is, for example, parallel or essentially parallel (an angle difference of less than 5 degrees or less than 10 degrees) to the longitudinal axis of the endoscope for which the light-conducting device is foreseen and configured. For example, at an angle of 90 degrees between the viewing angle and the longitudinal axis of a shaft of an endoscope for which the light-conducting device is foreseen and configured, the angle between the surface normals of the light inlet surface and of the light outlet surface of the light-conducting device is 90 degrees. In addition to a light outlet surface whose surface normal is not parallel to the surface normal of a light inlet surface of the light-conducting device, the light-conducting device can comprise one or more light outlet surfaces whose surface normals are parallel to the surface normal of the light inlet surface.

The foregoing remarks concerning surface normals of light inlet and outlet surfaces refer, in particular but not exclusively, to light-conducting devices in which the light propagation direction is directly downstream in the light path from a light inlet surface parallel to the surface normals of the light inlet surface and the light propagation direction is directly upstream in the light path from a light outlet surface parallel to the surface normals of the light outlet surface. Alternatively, the light-conducting device at the light inlet surface and/or at the light outlet surface can each comprise a beveling. With a beveling in the light-conducting device, the light propagation direction directly downstream in the light path from a light inlet surface is not parallel to the surface normals of the light inlet surface or the light propagation direction directly upstream in the light path from a light outlet surface is not parallel to the surface normals of the light outlet surface.

A light-conducting device as described here, with or without beveling on a light inlet surface or on a light outlet surface, can be configured for a light propagation direction directly downstream in the light path from the light inlet surface and a light propagation direction directly upstream in the light path from the light outlet surface, which are not parallel.

A light-conducting device as described here comprises in particular a cross-section that varies in the curved, rigid portion along the foreseen propagation path of illuminating light.

The propagation path of the illuminating light corresponds, depending on the case, to the path of the optic fibers in the light-conducting device. The cross-section of the light-conducting device can vary in the rigid portion, without, in any case, the cross-sections of the optic fibers varying. In particular with optic fibers with constant cross-section, the surface area of the cross-section of the light-conducting device can be constant in the rigid portion along the foreseen propagation path of illuminating light.

With a varying cross-section, the rigid portion of the light-conducting device can be adjusted to the structural space that is available, which typically varies in the longitudinal direction of the shaft, in particular close to the distal end of an endoscope.

A light-conducting device as described here, even before insertion of an inner shaft into an outer shaft for an endoscope, can be joined either with the inner shaft or with the outer shaft.

As already mentioned, the inner shaft is typically configured to include or to contain the observation beam path. The outer shaft of an endoscope is typically configured to include the inner shaft with observation beam path and the light-conducting device. The light-conducting device can be welded, or grouted at a temperature in the range of the melting temperature or of the glass transition or softening temperature of the material of the light-conducting device, with the outer surface of the inner shaft or with the inner surface of the outer shaft. It is thereby possible to form a mechanically particularly robust unit from the light-conducting device and the inner shaft or from the light-conducting device and the outer shaft, before the inner shaft is inserted into the outer shaft. Alternatively, the light-conducting device can be soldered by means of a metallic or non-metallic solder, cemented or cast with the inner shaft or with the outer shaft.

After insertion of the inner shaft, which already forms a mechanical unit with the light-conducting device, into the outer shaft or of the inner shaft into the outer shaft, which already forms a mechanical unit with the light-conducting device, the inner shaft and outer shaft can be connected with one another by metallurgical bonding and/or force locking and/or form locking. Thereafter, the light-conducting device, which, even before insertion of the inner shaft into the outer shaft, forms a mechanical unit with one of the two shafts, is mechanically connected both with the inner shaft and with the outer shaft, directly or indirectly in each case.

With a light-conducting device as described here, the light inlet surface of the light-conducting device can be foreseen and configured to be positioned on the proximal end of an endoscope.

If the light-conducting device includes a number of optic fibers, the light inlet surface of the light-conducting device is configured by the light inlet surfaces of the optic fibers. The light inlet surface of the light-conducting device is configured, in particular, to be coupled on the proximal end of an endoscope with a light source integrated in the proximal end of the endoscope or with an external light source by means of a light-conducting cable.

The light-conducting device can be of rigid configuration, from the light inlet surface foreseen and configured on the proximal end of an endoscope all the way to the light outlet surface foreseen on the distal end of the endoscope. Alternatively, the light-conducting device can comprise one or more flexible portions. If the light-conducting device includes a number of optic fibers, then they are joined with one another, in particular, only in a short curved, rigid portion, which connects to the light outlet surface of the light-conducting device, so that the light-conducting device downstream from the curved, rigid portion in the light path is flexible because of the flexibility of the individual optic fibers, which are not joined together.

An at least partly flexible configuration of the light-conducting device can prevent the development of mechanical tensions because of different thermal expansion coefficients, for example in autoclaving the finished endoscope, and thus can increase the lifetime of an endoscope that is produced with the light-conducting device.

With a light-conducting device as described here, the light inlet surface can be foreseen and configured to be positioned at a location other than on the proximal end of an endoscope, in particular in the shaft of the endoscope.

In particular, the light inlet surface is foreseen and configured to be positioned close to the distal end of an endoscope, such that the light-conducting device is, in particular, configured as completely rigid or the curved, rigid portion includes the entire light-conducting device. The light inlet surface of the light-conducting device in this case is foreseen to be optically coupled directly with a light source close to the distal end of the endoscope, or with a light source on the proximal end of the endoscope by means of an additional light conductor, or with a separate light source by means of an additional light conductor and of a light-conducting cable that can be removed from the endoscope. The light inlet surface of the light-conducting device can be in particular cemented or metallurgically bonded by means of a transparent material with a light outlet surface of the additional light conductor.

A light-conducting device as described here comprises in particular several light outlet surfaces, which are configured for a light outlet in different solid angles.

In particular, the surface normals of light outlet surfaces of the light-conducting device form angles of at least 15 degrees or at least 30 degrees. With several light outlet surfaces, the light-conducting device is capable—depending on the switching of illuminating light into one or more light inlet surfaces of the light-conducting device—or radiating illuminating light simultaneously or in alternation into several separate or overlapping solid angles. Thus the light-conducting device is suited in particular for an endoscope with adjustable viewing angle.

A light-conducting device as described here includes in particular several bundles of optic fibers, each with a rigid portion.

The bundles of optic fibers can be mechanically rigidly connected with one another and thus, in particular, can comprise a common light inlet surface or several separate light inlet surfaces and a common light outlet surface or several light outlet surfaces separated from one another. In particular, the rigid portions, one of which at least is curved in portions, are joined together. Alternatively, the bundles of optic fibers are not mechanically connected, but in their spatial configuration they are attuned to one another. Thus the bundles of optic fibers can be produced separately.

Several bundles of optic fibers make possible, especially with separate light outlet surfaces, an alternative or simultaneous radiation of illuminating light into different solid angles.

An endoscope includes a light-conducting device as described here.

The endoscope is configured in particular for a viewing angle that is not parallel to the longitudinal axis of the endoscope.

For example, the endoscope is configured for one or more fixed viewing angles, which form angles of 30 degrees, 45 degrees, 60 degrees and 90 degrees with the longitudinal axis of the shaft of the endoscope. Alternatively, the endoscope is configured, for example, for a continuous spectrum of viewing angles that form angles with the longitudinal axis of the shaft of the endoscope in a predetermined angle range, such that the angle range extends, for example, from zero degrees to 120 degrees or from 45 degrees to 120 degrees.

With an endoscope as described here, the light-conducting device is joined either with an outer shaft or with an inner shaft of the endoscope.

In particular, the light-conducting device is not joined either with the inner shaft or with the outer shaft.

With a method to produce a light-conducting device for an endoscope to conduct illuminating light to the distal end of the endoscope, a curved, rigid portion of the light-conducting device is generated in which the light-conducting device has a predetermined spatial configuration, so that the curved, rigid portion is foreseen for positioning on a distal end of an endoscope, so that the step of generating is executed at least either before insertion of the light-conducting device into an endoscope or before a direct or indirect mechanical connection of the light-conducting device with an inner shaft for an endoscope or before a direct or indirect mechanical connection of the light-conducting device with an outer shaft for an endoscope.

With a method as described here, the step of generating a curved, rigid portion includes, in particular, positioning, curving and joining of optic fibers in a portion in a predetermined curved spatial configuration, such that the portion in which the optic fibers are joined with one another and curved is foreseen for positioning on a distal end of an endoscope.

The steps of positioning, curving and joining can be executed in the indicated sequence or in another sequence or at least partly simultaneously. The optic fibers are joined, in particular, by welding or by pressing at a temperature in the range of the melting temperature or the glass transition or softening temperature of the material of the optic fibers. Alternatively, the optic fibers can be soldered by means of a metallic or non-metallic solder, cemented or cast.

With a method to produce a light-conducting device as described here, a lighting device as described here is produced, in particular.

With a method to produce an endoscope, a light-conducting device as described here or a light-conducting device produced by a method as described here is inserted into a shaft for an endoscope.

The method to produce an endoscope can include further steps, for example the production of a light-conducting device according to the methods described above.

### BRIEF DESCRIPTION OF THE DRAWINGS

Embodiments are described in greater detail hereinafter with reference to the appended drawings, which are as follows:

FIG. 1 shows a schematic depiction of an endoscope.

FIG. 2 shows a schematic depiction of an additional endoscope.

FIG. 3 shows an additional schematic depiction of a light-conducting device from FIG. 2.

FIG. 4 shows a schematic depiction of a first cross-section of the light-conducting device from FIG. 3.

FIG. 5 shows a schematic depiction of a second cross-section of the light-conducting device from FIG. 3.

FIG. 6 shows a schematic depiction of a third cross-section of the light-conducting device from FIG. 3.

FIG. 7 shows a schematic flow diagram of a method to produce an endoscope.

### DETAILED DESCRIPTION OF THE INVENTION

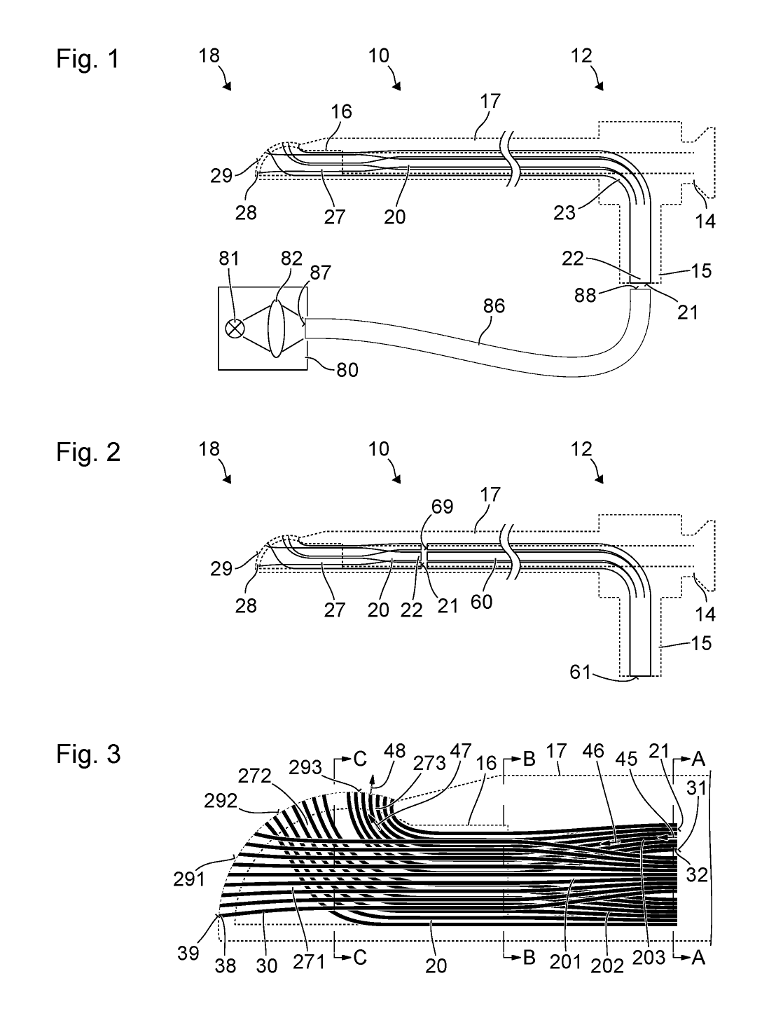

FIG. 1 shows a schematic depiction of an endoscope 10 with a light-conducting device 20. Because the light-conducting device 20 and its characteristics are the focal point of this application and can be produced or generated independently of the endoscope 10, said endoscope 10 is depicted in dotted outline.

The endoscope 10 comprises a proximal end 12 with an eyepiece 14 and a coupling 15 for a light-conducting cable, and a distal end 18. Extending between the proximal end 12 and the distal end 18 is an outer shaft 17, in which the light-conducting device 20 and an inner shaft 16 are positioned with an observation beam path that is not described any further here. The inner shaft 16 extends from the distal end 18 to the proximal end 12 of the endoscope and has a cross-section that is constant over a large area. At the distal end 18 of the endoscope 10, the inner shaft 16 and the outer shaft 17 each have non-constant cross-sections, which are required in particular for devices for an adjustable viewing angle of the endoscope 10.

A light source device 80 with a light source 81 and an imaging device 82 is coupled with the coupling 15 on the proximal end 12 of the endoscope 10. The imaging device 82, in particular an objective lens or one or more lenses or mirrors, projects the light source 81 onto a light inlet surface 87 of the light-conducting cable 86. A light outlet surface 88 of the light-conducting cable 86 is held by mechanical coupling devices on the light-conducting cable 86 and on the coupling 15 on the endoscope 10 opposite a light inlet surface 21 of the light-conducting device 20 in the endoscope 10. The coupling devices on the light-conducting cable 86 and on the coupling 15 are not shown in FIG. 1. The light outlet surface 88 of the light-conducting cable 86 and the light inlet surface 21 of the light-conducting device 20 are shown in FIG. 1 at a distance from one another. In fact, they can be pressed against one another by the aforementioned coupling devices.

The light-conducting device 20 is curved several times from its proximal end 22 on its light inlet surface 21 to its distal end 28 with several light outlet surfaces 29 and has a varying cross-section. It can be recognized in particular in FIG. 1 that the light-conducting device 20 downstream in the light path from the coupling 15 is split into several strands or bundles, which run parallel for long distances and partially cross one another close to the distal end 18 of the endoscope 10.

The light-conducting device 20 can comprise a flexible portion 23, which includes in particular a curvature between the coupling 15 and the outer shaft 17 of the endoscope 10. The flexible portion 23 can extend nearly to the distal end 18 of the endoscope 10, in particular approximately in the entire area in which the light-conducting device 20 is shown straight in FIG. 1. Alternatively, the entire light-conducting device 20 can be of rigid configuration.

FIG. 2 shows a schematic depiction of an additional endoscope with an additional light-conducting device 20, which resembles in some characteristics the light-conducting device described above with reference to FIG. 1. The light-conducting device 20 shown in FIG. 2 is shorter than the light-conducting device described above with reference to FIG. 1. In particular, the proximal end 22 and the light inlet surface 21 of the light-conducting device 20 are not positioned on the proximal end 12 of the endoscope 10 in the area of the coupling 15, but rather in the outer shaft 17 close to the distal end 18 of the endoscope 10.

A light conductor 60 extends from the coupling 15 of the endoscope 10 to the proximal end 22 of the light-conducting device 20. A light inlet surface 61 of the light conductor 60 is positioned on the coupling 15 in such a way that it can be optically coupled with a light outlet surface of a light-conducting cable. A light outlet surface 69 of the light conductor 60 is positioned opposite the light inlet surface 21 of the light-conducting device 20. The light outlet surface 69 of the light conductor 60 and the light inlet surface 21 of the light-conducting device 20 are shown at a distance from one another in FIG. 2. In fact, for an optimal coupling, the light outlet surface 69 of the light conductor 60 and the light inlet surface 21 of the light-conducting device 20 can be positioned directly contiguous with one another, mechanically pressed against one another and/or mechanically and optically coupled with one another by an optical cement or other transparent material with appropriate optical properties.

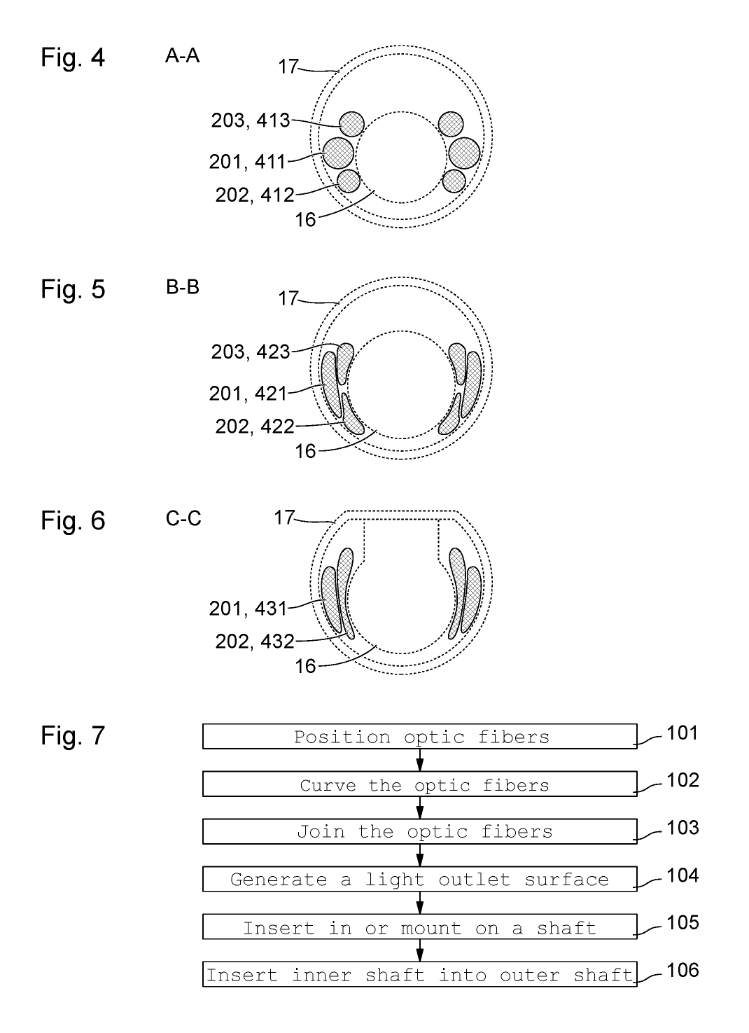

FIG. 3 shows another schematic depiction of the light-conducting device from FIG. 2. It can be recognized in the enlarged depiction in FIG. 3 that the light-conducting device 20 includes several bundles 201, 202, 203 of optic fibers 30. The optic fibers 30 within each bundle 201, 202, 203 are shown in FIG. 3 positioned alongside one another, essentially parallel and at the same distances. Different cross-sections of the bundles 201, 202, 203 are indicated by mutual distances of the optic fibers, varying in the longitudinal direction of the optic fibers 30 within a bundle 201, 202, 203. These different cross-sections of the bundles 201, 202, 203 are described more thoroughly below with reference to FIGS. 4 through 6. The schematic depiction in FIG. 3, however, is not to be interpreted to mean that the bundles 201, 202, 203 have quasi-one-dimensional cross-sections within which the optic fibers 30 are positioned exclusively in band form or alongside one another.

Light inlet surfaces 31 of the optic fibers 30 positioned within a surface, in particular within a plane, form the light inlet surface 21 of the light-conducting device 20. The light inlet surface 21 includes a number of non-connected partial surfaces that correspond to the number of bundles 201, 202, 203. Alternatively, contrary to the depiction in FIG. 3, the bundles 201, 202, 203 can be brought together in the area of the light inlet surface 21, so that the light inlet surfaces 31 of the optic fibers 30 of all bundles 201, 202, 203 form a single connecting light inlet surface 21.

The light outlet surfaces 39 on the distal ends 38 of the optic fibers 30 of a bundle 201, 202, 203 form the light outlet surface 291, 292, 293 of the corresponding bundle 201, 202, 203. In the example shown in FIG. 3, a first bundle 201 comprises close to its light outlet surface 291 an area 271 that is weakly curved, at most. A second bundle 202 and a third bundle 203, upstream in the light path from their light outlet surfaces 292, 293, comprise strongly curved, rigid areas 272, 273. In addition, the second bundle 202 and the third bundle 203, downstream in the light path from the light inlet surface 21, comprise weakly curved areas. A corresponding curvature of the optic fibers 30 of these bundles corresponds to the curvature of the second bundle 202 and of the third bundle 203.

The foreseen propagation paths of illuminating light inside the light-conducting device 20, in particular inside the individual bundles 201, 202, 203, correspond to the course or the direction of the individual optic fibers 30 inside the bundles 201, 202, 203. Arrows on the third bundle 203, by way of example, indicate the surface normal 45 of the light inlet surface 21, a light propagation direction 46 downstream in the light path from the light inlet surface 21, another light propagation direction 47 in the strongly curved, rigid area 273 and a center surface normal of the light outlet surface 293 of the bundle 203. The surface normals 45, 48 of the light inlet surface 21 or of the light outlet surface 293 correspond in the illustrated example essentially to the centrally foreseen light propagation directions inside the bundle 203 directly downstream in the light path from the light inlet surface 21 or directly upstream in the light path from the light outlet surface 293.

The light propagation direction in the example illustrated in FIG. 3 is at first essentially parallel to the longitudinal axis of the outer shaft 17 of an endoscope indicated in broken lines in FIG. 3, for which the light-conducting device 20 is foreseen (arrow 45). Downstream in the light path from the light inlet surface 21, the light propagation direction at first varies slightly (arrow 46), so that the third bundle 203 partly crosses the first bundle 201. Farther downstream in the light path, the light propagation direction is approximately constant for a brief distance, and the optic fibers run essentially parallel. Farther downstream in the light path, the light propagation direction varies as far as the light outlet surface 293 by more than 90 degrees (arrows 47, 48).

The individual bundles 201, 202, 203 of the light-conducting device 20 are rigid by means of welding, melting, soldering, cementing or casting of the individual fiber optics 30, in particular from the light inlet surface 21 to the light outlet surfaces 291, 292, 293. The bundles 201, 202, 203 can be rigidly combined with one another mechanically or configured as separate components.

FIGS. 4 through 6 show schematic depictions of sections along the sectional planes A-A, B-B or C-C indicated in FIG. 3 through the light-conducting device 20 from FIGS. 2 and 3. The sectional planes A-A, B-B and C-C are each perpendicular to the planes of projection of FIGS. 2 and 3. The contours of the inner shaft 16 and of the outer shaft 17 are indicated in dotted lines. While only three bundles 201, 202, 203 of optic fibers are shown in FIG. 3, in each of FIGS. 4 through 6 it is possible to recognize the cross-sections of six bundles, which are positioned symmetrically to one another by pairs on two opposite sides of the inner shaft 16. Three bundles 201, 202, 203 positioned close to one another on one side of the inner shaft in each case form a light-conducting device 20. Alternatively, all six bundles together form a light-conducting device 20. Hereinafter, because of the symmetry, reference is made only to three bundles 201, 202, 203 positioned close to one another on one side of the inner shaft 16.

Alternatively, contrary to the depiction in FIGS. 4 through 6, two light-conducting devices positioned on opposite sides of the inner shaft 16 can be configured asymmetrically to one another. For example, a first light-conducting device on one side of the inner shaft comprises bundles and light outlet surfaces to radiate illuminating light at angles of zero degrees, 48 degrees and 96 degrees, and a second light-conducting device on an opposite side of the inner shaft comprises bundles and light outlet surfaces to radiate illuminating light at angles of 24 degrees, 72 degrees and 120 degrees.

Alternatively to the depiction in FIGS. 4 through 6, it is possible on only one side of the inner shaft to position a light-conducting device that can include one, two, three or more bundles of optic fibers.

In the plane A-A shown in FIG. 4, each bundle 201, 202, 203 (compare FIG. 3) has an essentially circular-shaped cross-section 411 or 412 or 413. Alternatively and contrary to the depiction in FIG. 4, all bundles 201, 202, 203 in the plane A-A can be combined into one bundle with a single connecting cross-section. In the plane B-B shown in FIG. 5, each bundle 201, 202, 203 has a flattened and elongated cross-section 421 or 422 or 423. Consequently, the bundles 201, 202, 203, despite the enlarged cross-section of the inner shaft 16 in the area of the plane B-B, find space at its sides. The plane C-C does not intersect the third bundle 203. The cross-section 432 of the second bundle 202, which is strongly curved in the area of the plane C-C, is particularly distended in lengthwise direction, so that the entire second bundle 202 passes through the narrow space that is available between the inner shaft 16 and the first bundle 201. From a comparison of FIGS. 3 and 6, it can be recognized that the light propagation direction and the longitudinal axes of the optic fibers 30 of the second bundle 202 in the area of plane C-C are not perpendicular to the plane C-C.

FIG. 7 shows a schematic flow diagram of a method to produce an endoscope. Although the method is also suitable to produce an endoscope with a light-conducting device whose characteristics differ from the characteristics described above with reference to FIGS. 1 through 6, reference numbers from FIGS. 1 through 6 are used below by way of example in order to make the description more understandable.

In a first step 101, optic fibers 30 are positioned in one or more bundles 201, 202, 203. In a second step 102, the optic fibers 30 are curved into a predetermined spatial configuration, as can be seen for example in FIGS. 3 through 6. In a third step 103, the optic fiber 30 are joined together, in particular at a temperature close to the melting temperature or the glass transition or softening temperature of the material of the optic fibers 30, or are pressed together, soldered by means of a metallic or non-metallic solder, cemented or cast.

The optic fibers 30, particularly as shown in FIG. 7, are at first positioned, then curved and then joined. The sequence of these steps can be partly modified, however. In addition, the steps can be partly executed simultaneously. In particular, the optic fibers can be curved when positioned in a corresponding negative shape. In addition, the optic fibers can be curved during the joining, that is, for example, at first positioned in one or more bundles 201, 202, 203, then heated to a temperature close to the melting temperature or the glass transition or softening temperature, brought into the predetermined spatial configuration and pressed into it and thus melted or welded.

The steps of positioning 101, curving 102 and joining 103 can be executed individually for each bundle 201, 202, 203 of a light-conducting device, or executed simultaneously for several or all bundles 201, 202, 203. In the process or thereafter, the bundles 201, 202, 203 can be joined together. With the steps of positioning 101, curving 102 and joining 103, a curved, rigid portion 272, 273 of a light-conducting device 20 is generated.

In a fourth step 104, one or more light outlet surfaces 29, 291, 292, 293 can be generated, for example by grinding and polishing the distal end or ends.

The steps of positioning 101, curving 102 and joining 103, and optionally the step of generating 204 a light outlet surface 104, together form a method to produce a light-conducting device 20 that can be executed at separate times and places from, and logistically independently of, other steps in the method for producing an endoscope 10.

In a fifth step 105, the light-conducting device produced in the preceding steps is inserted into an outer shaft 17 or mounted on an inner shaft 16, in particular joined with the outer shaft 17 or the inner shaft 16. Joining of the light-conducting device 20 with an inner shaft 16 or with an outer shaft 17, in addition, can occur simultaneously with the joining 103 of the optic fibers to one another.

In a sixth step 106, the inner shaft 16 is inserted into the outer shaft 17. Here, depending on the execution of the fifth step 105, the inner shaft 16 can be inserted with the light-conducting device 20 into the outer shaft 17 or the inner shaft 16 can be inserted into the outer shaft 17 that is equipped with the light-conducting device 20.

1-11. (canceled)

12. A method for producing a light-conducting device for an endoscope to conduct illuminating light to the distal end of the endoscope, comprising the steps of:
generating a curved, rigid portion in a plurality of optic fibers with a predetermined spatial configuration,
the curved, rigid portion located within a distal end of an endoscope between an inner shaft and an outer shaft of the endoscope, such that the generating step is executed at least either before insertion of the light-conducting device into the endoscope or before a direct or indirect mechanical connection of the light-conducting device with the inner shaft of the endoscope or before a direct or indirect mechanical connection of the light-conducting device with the outer shaft of the endoscope; and
wherein the light-conducting device has a cross-section that varies in the curved, rigid portion along a propagation path of illuminating light.

13. The method of claim 12, wherein the step of generating the curved, rigid portion comprises the following steps:
positioning the optic fibers;
curving the optic fibers; and
joining the optic fibers in a portion in a predetermined curved spatial configuration,
wherein the portion in which the optic fibers are joined together and curved is for positioning on a distal end of an endoscope.

14. The method of claim 13, wherein the optic fibers each have a cross-section with an area that varies in a light propagation direction.

15. A method of producing an endoscope, having the following step:
insertion of a light-conducting device, produced according to claim 12 into an outer shaft.

16. A method of producing an endoscope, comprising the steps of:
generating a light-conducting device having a curved, rigid portion with a predetermined spatial configuration;
after the generating step, inserting the light-conducting device into the shaft of an endoscope; and
after the inserting step, coupling the light-conducting device with the shaft of an endoscope.

17. The method of claim 16, further comprising the step of:
arranging several bundles of optic fibers in the light-conducting device to direct illuminating light in a light propagation direction between a proximal end and a distal end of the endoscope, the several bundles of optic fibers each having a respective cross-sectional area that varies in the light propagation direction.

18. The method of claim 17, wherein the arranging step includes:
positioning the several bundles of optic fibers in the light-conducting device such that the several bundles of optic fibers at least partially cross one another in a first section of the light-conducting device close to the distal end of the endoscope, and the several bundles of optic fibers run parallel to one another in a second section of the light conducting device farther from the distal end of the endoscope than the first section.

19. The method of claim 16, wherein the generating step includes:
positioning several bundles of optic fibers in the light-conducting device;
curving the several bundles of optic fibers;
joining the several bundles of optic fibers in a portion in the predetermined spatial configuration in order to form the curved, rigid portion.

20. The method of claim 16, wherein the inserting step includes:
positioning the light-conducting device such that the curved rigid portion is located within the distal end of the endoscope between an inner shaft and an outer shaft of the endoscope.

21. The method of claim 20, wherein the inserting step further includes:
positioning the light-conducting device in the inner shaft of the endoscope.

22. The method of claim 20, wherein the inserting step further includes:
positioning the light-conducting device in the outer shaft of the endoscope.

23. The method of claim 21, wherein the coupling step includes:
coupling the light-conducting device to the inner shaft of the endoscope.

24. The method of claim 22, wherein the coupling step includes:
coupling the light-conducting device to the outer shaft of the endoscope.
